(19) United States
(12) Patent Application Publication
(10) Pub. No.: US 2009/0234017 A1
(43) Pub. Date: Sep. 17, 2009

(54) TREATING SKIN DISORDERS

(76) Inventors: Evangeline L. Stookey, Vinita, OK (US); Paola Sandroni, Rochester, MN (US); Mark R. Pittelkow, Rochester, MN (US)

Correspondence Address: FISH & RICHARDSON P.C., PO BOX 1022, MINNEAPOLIS, MN, 55440-1022, US

(21) Appl. No.: 12/468,716

(22) Filed: May 19, 2009

Related U.S. Application Data
(62) Division of application No. 11/415,571, filed on May 2, 2006.
(60) Provisional application No. 60/725,507, filed on Oct. 11, 2005.

(51) Int. Cl. A61K 31/165; A61K 31/137; A61P 17/06

(52) U.S. Cl. 514/626; 514/653

(57) ABSTRACT

This document relates to methods and materials involved in treating skin disorders. For example, methods and materials for using an alpha adrenergic receptor agonist (e.g., midodrine) to treat mammals having a skin disorder are provided.

### CROSS-REFERENCE TO RELATED APPLICATIONS

This application is a divisional of U.S. application Ser. No. 11/415,571, filed May 2, 2006, which claims the benefit of U.S. provisional application Ser. No. 60/725,507, filed Oct. 11, 2005. The disclosures of the prior applications are considered part of (and are incorporated by reference in) the disclosure of this application.

### BACKGROUND

1. Technical Field

This document relates to methods and materials involved in treating mammals having a skin disorder such as psoriasis. For example, this document provides methods and materials for treating skin disorders using an alpha adrenergic receptor agonist (e.g., midodrine).

2. Background Information

Skin is the largest organ in the body and is the first line of defense against dirt, microorganisms, and other foreign objects. Skin disorders number in the hundreds and are common throughout the world. Most skin disorders, such as acne, eczema, and psoriasis, display symptoms on the surface of the skin. Causes of skin disorders include infection, sun exposure, allergies, hormones, pregnancy, and genetics. Skin disorders can cause physical discomfort, anxiety, depression, and a lack of self-confidence. Skin disorders can lead to social isolation if obviously visible. Certain dermatoses, such as allergic hand eczema in a builder or hairdresser, can interfere with or even prevent working.

### SUMMARY

This document provides materials and methods that can be used to treat skin disorders. For example, this document provides compositions containing alpha adrenergic receptor agonists (e.g., midodrine) as well as methods for using such compositions to treat mammals having skin disorders. As described herein, a composition containing an alpha adrenergic receptor agonist can be administered topically to a mammal (e.g., a human) having a skin disorder under conditions wherein the skin disorder is treated. For example, a composition provided herein can be used to reduce the severity of one or more symptoms of a skin disorder or can be used to completely resolve the skin disorder. The methods and materials provided herein can allow clinicians to treat patients having a skin disorder, thereby improving the patients' health and quality of life.

In general, one aspect of this document features a method for treating a mammal having a skin disorder. The method comprising, or consists essentially of, topically administering a composition comprising an alpha adrenergic receptor agonist to the mammal under conditions wherein the severity of a symptom of a skin disorder is reduced. The mammal can be a human. The skin disorder can be psoriasis, acne rosacea, lichen planus, or dermatitis. The alpha adrenergic receptor agonist can be midodrine. The alpha adrenergic receptor agonist can be desglymidodrine. The alpha adrenergic receptor agonist can be methoxamine or phenylephrine. The composition can comprise midodrine and desglymidodrine. The composition can comprise midodrine and one or more agents selected from the group consisting of desglymidodrine, steroids, vitamin D3, vitamin D3 analogs, anthralin, coal tar, retinoids, keratolytic agents, and tacrolimus. The symptom can be erythema, scaling, pruritus, or induration. The severity of the symptom can be reduced by at least 25 percent compared to the baseline condition. The severity of the symptom can be reduced by at least 50 percent compared to the baseline condition. The severity of the symptom can be reduced by at least 75 percent compared to the baseline condition. The severity of the symptom can be reduced by at least 95 percent compared to the baseline condition.

In another embodiment, this document features a method for treating a human having dermatitis. The method comprising, or consists essentially of: (a) identifying a human as having dermatitis, (b) topically administering an ointment composition comprising between 0.5% and 25% midodrine to the human under conditions wherein the severity of a symptom of the dermatitis is reduced, and (c) monitoring the human for the reduction in the severity of the symptom.

In another aspect, this document features a composition for topical administration comprising, or consisting essentially of, midodrine, an excipient, and an agent selected from the group consisting of desglymidodrine, methoxamine, phenylephrine, steroids, vitamin D3, vitamin D3 analogs, anthralin, coal tar, retinoids, keratolytic agents, and tacrolimus. The excipient can be selected from the group consisting of emulsifying agents, antioxidants, buffering agents, preservatives, humectants, penetration enhancers, chelating agents, gelforming agents, skin protective agents, and ointment bases.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides methods and materials for treating skin disorders. For example, this document provides methods for treating mammals having a skin disorder using an alpha adrenergic receptor agonist (e.g., midodrine). This document also provides compositions (e.g., topical compositions) containing alpha adrenergic receptor agonists that can be used to treat skin disorders.

Any type of mammal having a skin disorder can be treated using the methods and materials provided herein. For example, a human can be treated for a skin disorder. In some cases, a dog, cat, rat, mouse, horse, goat, sheep, cow, or monkey can be treated for a skin disorder.

A skin disorder can be any condition of the skin involving vascular dilatation and inflammation. For example, a skin disorder can be, without limitation, psoriasis (e.g., plaque psoriasis, guttate psoriasis, pustular psoriasis, inverse psoriasis, erythrodermic psoriasis, or psoriatic arthritis), acne rosacea, lichen planus, pityriasis rubra pilaris, telangiectasia, dermatitis (e.g., atopic dermatitis, nummular or discoid dermatitis, asteatotic dermatitis, stasis dermatitis, or seborrheic dermatitis), angiomas, hemangiomas, or vascular dilatation anomalies. In some cases, a skin disorder can be a skin disorder that is not related to skin cancer or not caused by a viral, bacterial, or fungal infection.

Any method can be used to determine whether or not a mammal has a skin disorder. For example, imaging technologies, such as radiography, can be used to determine whether or not a mammal has a vascular anomaly. In some cases, histologic diagnosis can be used to determine whether or not a mammal has a skin disorder. In some cases, the skin can be examined to determine whether or not a mammal has a skin disorder. Typically, a particular skin disorder can be identified based on the location and appearance of observable lesions. For example, plaque psoriasis typically includes the presence of sharply demarcated chronic erythematous plaques covered by silvery white scales that commonly appear on the elbows, knees, scalp, umbilicus, and lumbar area. Guttate psoriasis typically includes the presence of drop-shaped scaly macules on the trunk, arms, legs, and scalp. Pustular psoriasis typically includes the presence of white pustules (blisters of noninfectious pus) surrounded by red skin. Inverse psoriasis typically includes the presence of smooth patches of red, inflamed skin mainly in the armpits, groin, under the breasts, and around the genitals. Erythrodermic psoriasis typically includes the presence of a wide area of red and scaling skin. Psoriatic arthritis typically includes the presence of inflamed, scaly skin, as well as pitted, discolored nails and swollen, painful joints.

Once a mammal is determined to have a skin disorder, the mammal can be treated with an alpha adrenergic receptor agonist. Any alpha adrenergic receptor agonist such as midodrine, desglymidodrine, methoxamine, or phenylephrine can be used. In some cases, a combination of one, two, three, or more alpha adrenergic receptor agonists can be used to treat a skin disorder. For example, a combination of midodrine and desglymidodrine can be used to treat psoriasis.

In general, a composition containing at least one alpha adrenergic receptor agonist is administered to a mammal having a skin disorder under conditions wherein the severity of at least one symptom of the skin disorder is reduced. A composition containing an alpha adrenergic receptor agonist (e.g., midodrine) can contain any amount of an alpha adrenergic receptor agonist and can be in a form for any type of administration including, without limitation, oral, topical, or injections. For topical administration, a composition provided herein can be administered directly to the affected areas of the skin. In addition, a composition for topical administration can contain between 0.1% and 30% alpha adrenergic receptor agonist (e.g., midodrine) and can be in the form of a gel, cream, or lotion.

A composition containing an alpha adrenergic receptor agonist can include other components such as a steroid, tar (e.g., coal tar), anthralin, vitamin D3, a vitamin D3 analog (e.g., calcipotriene), a retinoid, a keratolytic agent, or tacrolimus. Examples of steroids that can be included in a composition containing an alpha adrenergic receptor agonist include, without limitation, desondide, flumethasone pivalate, fluocinolone acetonide, alclometasone dipropionate, hydrocontisone, desonide, hydrocortisone valerate, clocortolone pivalate, fluticasone propionate, halobetasol propionate, clobetasol propionate, betamethasone, diflorasone diacetate, and mometasone furoate. A retinoid can be any vitamin A derivative. For example, a retinoid can be tazarotene. A keratolytic agent can be any agent with keratolytic activity. For example, a keratolytic agent can be salicylic acid. In some cases, a keratolytic agent can be a preparation containing urea or an alpha-hydroxy acid, such as glycolic acid or lactic acid. In one embodiment, a composition containing an alpha adrenergic receptor agonist can be formulated for topical administration and can include one or more of the following components: between 0.0005% and 3.5% steroid, between 0.1% and 20% tar, between 0.01% and 7% anthralin, between 0.001% and 0.05% vitamin D3 or a vitamin D3 analog, between 0.01% and 0.5% retinoid, between 1% and 30% keratolytic agent, or between 0.01% and 0.5% tacrolimus.

The components of a composition provided herein can be obtained using common methods such as chemical synthesis, isolation from a natural source, refinement of a product isolated from a natural source, or a combination thereof.

As described herein, a composition containing at least one alpha adrenergic receptor agonist can be used to treat a skin disorder. Various factors can influence the actual effectiveness of a composition provided herein such as the use of multiple treatment agents and the severity of the skin disorder. The amount of a composition (e.g., a topical composition) administered or the frequency or duration of administration can be increased or decreased to adjust the efficacy of a composition containing a particular concentration of one or more alpha adrenergic receptor agonists. In some cases, a composition containing a higher or lower concentration of one or more alpha adrenergic receptor agonists can be used. In addition, the amount of a composition administered, the concentration of alpha adrenergic receptor agonist(s), or the frequency or duration of administration can be adjusted according to any side effects that may occur. Side effects can vary for each particular mammal and depend on multiple factors including, without limitation, the mammal's disease state, age, and lifestyle.

The frequency of administration of a composition (e.g., a topical composition) can be any frequency that reduces the severity of a symptom of a skin disorder without producing significant side effects. For example, a topical composition can be administered once daily, twice daily, every other day, once a week, or as needed. In addition, the frequency of administration can remain constant or can be variable during the duration of treatment. Various factors can influence the actual frequency of administration used for a particular application. For example, the concentration of alpha adrenergic receptor agonist(s), duration of treatment, use of multiple alpha adrenergic receptor agonists, occurrence of side effects, and severity of the skin disorder may require an increase or decrease in administration frequency.

An effective duration for administering a composition provided herein can be any duration that reduces the severity of a symptom of a skin disorder without producing significant side effects. Thus, the effective duration can vary from several days to several weeks, months, or years. In general, the effective duration for the treatment of a skin disorder can range in duration from several days to several months. In some cases, an effective duration can be for as long as an individual mammal is alive. A course of treatment can include rest periods. For example, a topical composition can be administered for 12 weeks followed by a 6-week rest period, and this treatment regimen can be repeated multiple times. Multiple factors can influence the actual effective duration used for a particular treatment. For example, an effective duration can vary with the frequency of administration, concentration of alpha adrenergic receptor agonist(s), use of multiple alpha adrenergic receptor agonists, occurrence of side effects, and severity of the skin disorder.

A topical composition useful for treating a skin disorder can be any form of topical composition. For example, a topical composition can be a cream, gel, ointment, spray, lotion, scalp lotion, shampoo, solution, powder, or hard paste. A topical formulation can also be used to impregnate an occlusive tape, which can then be used to treat a skin disorder.

In addition to containing at least one alpha adrenergic receptor agonist, a composition (e.g., a topical composition) can contain one or more pharmaceutically acceptable excipients. Excipients can include, without limitation, emulsifying agents, antioxidants, buffering agents, preservatives, humectants, penetration enhancers, chelating agents, gelforming agents, skin protective agents, and ointment bases.

After administering a composition provided herein to a mammal having a skin disorder, the mammal can be monitored to determine whether or not a symptom of the skin disorder improves compared to the baseline condition. Clinical symptoms can also be assessed to monitor the occurrence of side effects. A symptom of a skin disorder can include erythema, scaling, thickness (e.g., induration, plaque elevation), pruritus, a blister, a crust, a wheal, lichenification, a papule, swelling, a discoloration, a pustule, or visibly dilated blood vessels. A symptom can be monitored over time, such as before, during, and after treatment. Skin lesions can be photographed at baseline to aid in assessing changes in a symptom in response to treatment. A symptom scale, such as the Skindex-29 (Chren et al., Arch Dermatol 133:1440-3 (1997); Chren et al., J Cutan Med Surg 5:105-10 (2001)), can also be useful in monitoring symptoms of a skin disorder. Improvement of a symptom compared to the baseline condition can be slight (1%-24% improvement), fair (25%-49% improvement), good (50%-74% improvement), excellent (75%-99% improvement), or cleared (100% improvement).

The invention will be further described in the following examples, which do not limit the scope of the invention described in the claims.

### EXAMPLES

### Example 1—Treating Psoriasis

A 58 year old white female was treated for hypotension using midodrine. The dose of midodrine was five mg three times a day for three months. The dose was less than the standard therapy for hypotension, which is often 10 mg of midodrine three times a day. The patient also had moderate psoriasis. The patient's psoriasis was observed to improve dramatically (90% or more improvement), while the patient was taking midodrine. When the patient discontinued use of midodrine, the patient's psoriasis returned.

### Example 2—Treating Psoriasis in a Murine Model using a Topical Formulation of Midodrine

Twenty transgenic mice overexpressing amphiregulin in the epidermis and exhibiting a psoriasis-like skin phenotype are divided into experimental and control groups, with ten mice in each group. Both ears and the tail of each mouse are treated according to the following protocol. Mice in the experimental group are treated with a topical formulation containing 1 to 20 percent midodrine twice daily for four or eight weeks. Mice in the control group are treated in a similar manner with the vehicle lacking midodrine. After four weeks of treatment, five mice from each group are sacrificed. Portions of the ears and tails of the sacrificed mice are cryopreserved for additional studies that may be indicated. The remaining portions of the ears and tails are fixed in formalin, imbedded in paraffin, sectioned, and stained using hematoxylin and eosin (H&E) and immunocytochemical stains. The stained tissue sections are examined for epidermal and dermal changes associated with psoriasis, and the inflammatory infiltrate and blood vessel density are quantified. Sections from the experimental group are compared to those from the control group. After eight weeks of treatment, the remaining five mice in each group are sacrificed and analyzed in the same manner. A blanching assay also is performed to determine, by visual observation, whether or not inflamed, reddened areas blanch, or whiten. Affected areas, e.g. ears, of mice in the experimental group are compared with those of mice in the control group.

### Example 3—Treating Psoriasis using a Topical Formulation

A placebo-controlled, blinded, half-body study is conducted to confirm the response of psoriasis to treatment with a topical formulation of midodrine. Fifteen to twenty patients with chronic plaques covering 2 to 10 percent of their body surface area are enrolled in the study. The patients are subjected to a two week washout period to remove previous medications, such as topical steroids and Dovonex. Following the washout period, one to three plaques on one half of the body of each patient are treated with a topical formulation (e.g., cream or ointment) containing about 1.5 percent midodrine. One to three plaques on the other half of the body of each patient are treated with the vehicle lacking midodrine. Treatment of the left and right sides of the patients are randomized. The patients are treated twice daily for 12 to 16 weeks.

Individual lesions are scored based on erythema, scaling, induration, thickness, and pruritus (if present). Statistical analyses of the data are performed to evaluate differences between lesions that were treated with midodrine and lesions that were treated without midodrine. The patients are monitored for any local or systemic side effects, including worsening of the disease, pruritus, and evidence of dermatitis or other toxicity. Follow-up examinations are conducted four to six weeks after discontinuation of topical treatment with midodrine.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a human having psoriasis, said method comprising administering a composition comprising an alpha adrenergic receptor agonist to said human under conditions wherein the severity of a symptom of said psoriasis is reduced.

2. The method of claim 1, wherein said composition is administered topically to said human.

3. The method of claim 1, wherein said alpha adrenergic receptor agonist is midodrine.

4. The method of claim 1, wherein said alpha adrenergic receptor agonist is desglymidodrine.

5. The method of claim 1, wherein said alpha adrenergic receptor agonist is methoxamine or phenylephrine.

6. The method of claim 1, wherein said composition comprises midodrine and desglymidodrine.

7. The method of claim 1, wherein said composition comprises midodrine and one or more agents selected from the group consisting of desglymidodrine, steroids, vitamin D3, vitamin D3 analogs, anthralin, coal tar, retinoids, keratolytic agents, and tacrolimus.

8. The method of claim 1, wherein the symptom is erythema, scaling, pruritus, or induration.

9. The method of claim 1, wherein said severity of the symptom is reduced by at least 25 percent compared to a baseline condition.

10. The method of claim 1, wherein said severity of the symptom is reduced by at least 50 percent compared to a baseline condition.

11. The method of claim 1, wherein said severity of the symptom is reduced by at least 75 percent compared to a baseline condition.

12. The method of claim 1, wherein said severity of the symptom is reduced by at least 95 percent compared to a baseline condition.

13. The method of claim 1, wherein said method comprises identifying said human as having said psoriasis.

14. The method of claim 1, wherein said method comprises monitoring said human for said reduction in the severity of said symptom.
